(19) United States
(12) Patent Application Publication
(10) Pub. No.: US 2025/0281272 A1
(43) Pub. Date: Sep. 11, 2025

(54) OUTER EAR IMPLANT SYSTEMS, METHODS OF IMPLANTING, AND SELECTION KITS

(71) Applicant: PORIFEROUS, LLC, Newnan, GA (US)

(72) Inventors: Aaron Noble, Newnan, GA (US); Spencer Jones, Newnan, GA (US)

(21) Appl. No.: 19/075,458

(22) Filed: Mar. 10, 2025

Related U.S. Application Data
(60) Provisional application No. 63/563,869, filed on Mar. 11, 2024.

(51) Int. Cl. A61F 2/00; A61F 11/00

(52) U.S. Cl. CPC A61F 2/0059; A61F 11/00; A61F 2250/0064; A61F 2250/0097

(57) ABSTRACT

An ear implant selection kit is described including a set of semi-transparent or transparent contour templates, a separate set of gradation templates, and a set of three-dimensional ear implant models, all of which may be used to select an ear implant out of a set of implants that best matches the appearance of a contralateral ear of an individual. Related ear implant systems and methods are also described.

This application claims the benefit of U.S. Provisional Application Ser. No. 63/563,869, filed Mar. 11, 2024, titled “Outer Ear Implant Systems, Methods of Implanting, and Selection Kits,” the entire contents of which are hereby incorporated by reference.

### RELATED FIELDS

Implant systems, method of implanting, and selection kits for use in the same, in particular outer ear implant systems, outer ear implant implantation methods, and outer ear implant selection kits.

### BACKGROUND

Surgical reconstruction of an outer ear often uses a framework implant. The framework implant can be used to support the patient's overlying skin and generally provides a cartilage-like support structure, providing predictable aesthetic results. Porous polyethylene ear implants have been used for many years as a framework material. This material generally has good biocompatibility for reconstruction of ear malformations. Due to its porous structure, the material can promote revascularization at the implantation site.

The shape and size of ears vary widely from person to person. It is desirable for a selected ear implant to as closely match a patient's contralateral ear as reasonably possible. However, to date there remains a need for an effective solution for selecting the best match.

### SUMMARY

Outer ear implant systems, methods of selection, and selection kits may include a set of contour templates each of which corresponds in appearance to an outer ear implant of a set of outer ear implants, may further include a set of three-dimensional ear implant models corresponding in appearance to the outer ear implants, and may further include a set of gradation templates corresponding in appearance to the outer ear implants.

### FIRST EXAMPLE

In one example there is an outer ear implant selection kit for selecting an outer ear implant from a set of outer ear implants, the implant set including a first outer ear implant with a first appearance and a second outer ear implant comprising a second appearance, the first appearance being different from the second appearance. In this example the outer ear implant selection kit includes a set of templates corresponding to the set of outer ear implants, the set of templates including at least a first template and a second template. The first template corresponds in appearance to the first outer ear implant and the second template corresponds in appearance to the second outer ear implant.

In some implementations of this example the outer ear selection kit also includes a set of three-dimensional ear implant models including a first three-dimensional outer ear implant model that corresponds in appearance to the first outer ear implant and a second three-dimensional outer ear implant model that corresponds in appearance to the second outer ear implant.

In some implementations of this example the set of templates includes a set of semi-transparent or transparent contour templates in which the first template is a first contour template with a first side corresponding in appearance to the first outer ear implant, and in which the second template is a second contour template with a first side corresponding in appearance to the second outer ear implant.

In some implementations of this example the first contour template includes a label for the first outer ear implant and the second contour template includes a label for the second outer ear implant.

In some implementations of this example the first contour template includes first contour markings that, from the first side of the first contour template, correspond in appearance to contours of the first outer ear implant; and the second contour template includes second contour markings that are different from the first contour markings, the second contour markings, from the first side of the second contour template, correspond in appearance to contours of the second outer ear implant.

In some implementations of this example the contour markings of the first and second contour templates are opaque or semi-opaque contour lines.

In some implementations of this example the set of outer ear implants further includes a third outer ear implant that is a contralateral version of the first outer ear implant and a fourth outer ear implant that is a contralateral version of the second outer ear implant. In these implementations the ear selection kit may include a second side of the first contour template, in which the contour markings of the first contour template, from the second side of the first contour template, correspond in appearance to the third outer ear implant. In these implementations the ear selection kit may also include a second side of the second contour template, in which the contour markings of the second contour template, from the second side of the second contour template, correspond in appearance to the fourth outer ear implant.

In some implementations of this example the first side of the first contour template is labeled for the first outer ear implant and the second side of the first contour template is labeled for the third outer ear implant. And the first side of the second contour template is labeled for the second outer ear implant and the second side of the second contour template is labeled for the fourth outer ear implant.

In some implementations of this example the contour markings of the first contour template include an outer perimeter shape corresponding in appearance to an outer perimeter shape of the first outer ear implant; and the contour markings of the second contour template include an outer perimeter shape corresponding in appearance to an outer perimeter shape of the second outer ear implant.

In some implementations of this example the contour markings of the first contour template further includes at least one of: (i) a first helix contour marking corresponding in appearance to the first outer ear implant; (ii) a first concha contour marking corresponding in appearance to the first outer ear implant; and (iii) a first tragus contour marking corresponding in appearance to the first outer ear implant. Additionally, the contour markings of the second contour template may further include at least one of (i) a second helix contour marking corresponding in appearance to the second outer ear implant; (ii) a second concha contour marking corresponding in appearance to the second outer ear implant; and (iii) a second tragus contour marking corresponding in appearance to the second outer ear implant.

In some implementations of this example the outer ear selection kit further includes a set of gradation templates including: a first gradation template corresponding in appearance to the first outer ear implant, the first gradation template being labeled for the first outer ear implant; and a second gradation template corresponding in appearance to the second outer ear implant, the second gradation template being labeled for the second ear implant.

In some implementations of this example the first gradation template includes first depth shading that corresponds in appearance to the first outer ear implant; and the second gradation template includes second depth shading that is different from the first depth shading and that corresponds in appearance to the second outer ear implant.

In some implementations of this example the first depth shading of the first gradation template includes at least one of: (i) a first helix depth shading corresponding in appearance to a helix of the first outer ear implant; (ii) a first concha depth shading corresponding in appearance to a concha of the first outer ear implant; and (iii) a first tragus depth shading corresponding in appearance to a tragus of the first outer ear implant. Additionally the second depth shading of the second gradation template may include at least one of: (i) a second helix depth shading corresponding in appearance to a helix of the second outer ear implant; (ii) a second concha depth shading corresponding in appearance to a concha of the second outer ear implant; and (iii) a second tragus depth shading corresponding in appearance to a tragus of the second outer ear implant.

In some implementations of this example the first outer ear implant is a one piece implant including a tragus, a concha, a helix, and a conchal eminence; and the second outer ear implant is a one piece implant comprising a tragus, a concha, a helix, and a conchal eminence. An appearance of at least one of the tragus, concha, and helix of the first outer ear implant is different from an appearance of the tragus, concha, and helix of the second outer ear implant.

In some implementations of this example the set of outer ear implants is a set of outer ear framework implants.

### SECOND EXAMPLE

Another example is a method of selecting an outer ear implant for implantation on a person from a set of outer ear implants, the set of outer ear implants including several outer ear implants having different aesthetic appearances. In this example the method of selecting from the set of outer ear implants includes using a set of templates that includes templates corresponding in appearance to one of the outer ear implants, comparing at least one of the templates from the set of templates to a contralateral ear of the person. In this example, the method further includes, based on the comparison of the at least one template, selecting one of the outer ear implants from the set of outer ear implants for ipsilateral implantation on the person.

In some implementations of this example the method also includes, using a set of three-dimensional ear implant models each corresponding in appearance to one of the outer ear implants, trialing at least one of the three-dimensional ear implant models with the person. And, based on the comparison of the at least one template and based on the trialing of the at least one three-dimensional ear implant model, selecting one of the outer ear implants from the set of outer ear implants for ipsilateral implantation on the person.

In some implementations of this example the set of templates includes a set of semi-transparent or transparent contour templates, in which the comparison of the template to the contralateral ear of the person including positioning a contour template over the contralateral ear of the person.

In some implementations of this example the contour template positioned over the contralateral ear of the person includes contour markings indicating an outer perimeter shape corresponding in appearance to an outer perimeter shape of a first outer ear implants of the set of outer ear implants.

In some implementations of this example the contour template positioned over the contralateral ear of the person further includes additional contour markings including at least one of: (i) a helix contour marking corresponding in appearance to the first outer ear implant; (ii) a first concha contour marking corresponding in appearance to the first outer ear implant; and (iii) a first tragus contour marking corresponding in appearance to the first outer ear implant.

In some implementations of this example the method further includes, using a set of gradation templates including several gradation templates each corresponding in appearance to one of the outer ear implants, comparing at least one of the gradation templates to the contralateral ear of the person. Based on the comparison of the at least one contour template and based on the trialing of the at least one three-dimensional ear implant model and based on the comparison of the at least one gradation template, one of the outer ear implants from the set of outer ear implants may be selected for ipsilateral implantation on the person.

In some implementations of this example the outer ear implants of the set of outer ear implants each is a one piece implant including a tragus, a concha, a helix, and a conchal eminence.

In some implementations of this example the outer ear implants of the set of outer ear are each an outer ear framework implant.

### BRIEF DESCRIPTION OF FIGURES

FIG. 1 shows an example of a set of outer ear implants.

FIG. 2 shows views of a first ear implant of the set of ear implants of FIG. 1.

FIG. 3 shows views of a second ear implant of the set of ear implants of FIG. 1.

FIG. 4 shows views of a third ear implant of the set of ear implants of FIG. 1.

FIG. 5 shows views of a fourth ear implant of the set of ear implants of FIG. 1.

FIG. 6 shows enlarged views of the first ear implant of FIG. 1.

FIG. 7 shows an example of an ear implant selection kit.

FIG. 8 shows a set of contour templates of the ear implant sizing kit of FIG. 7.

FIG. 9 shows two sides of one of the contour templates of the set of contour templates of FIG. 7.

FIG. 10 shows a set of gradation templates of the ear implant sizing kit of FIG. 7.

FIG. 11 shows one of the gradation templates of the set of gradation templates of FIG. 10.

FIG. 12 shows a set of ear implant models of the ear implant sizing kit of FIG. 7.

FIG. 13 shows one of the ear implant models of the set of ear implant models of FIG. 12.

FIG. 14 shows two of the ear implants of the set of ear implants of FIG. 1, and the contour template, gradation template, and implant model corresponding to those two ear implants.

FIG. 15 shows an example of a method of implanting an ear implant.

FIG. 16 shows an example of comparing a contour template to a contralateral ear of an individual.

FIG. 17 shows an example of comparing an ear implant model to a contralateral ear of an individual.

### DETAILED DESCRIPTION

### Ear Implant Set

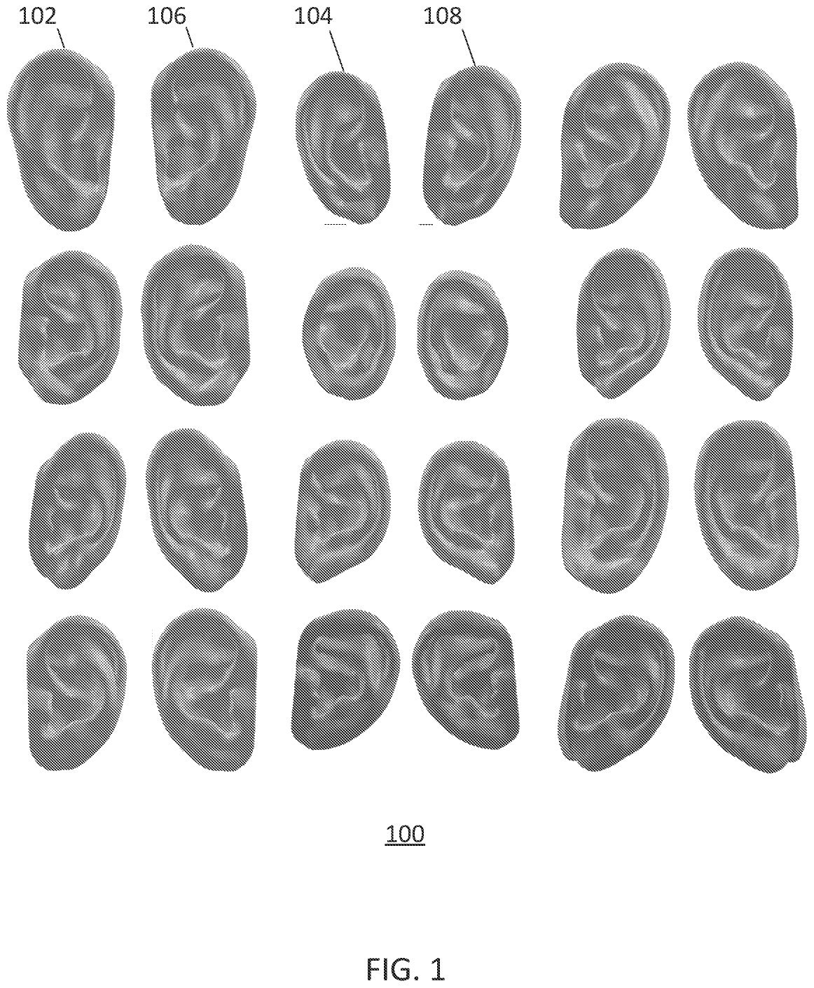

FIGS. 1-5 show an example of a set of ear implants 100 including a first ear implant 102, a second ear implant 104, a third ear implant 106, and a fourth ear implant 108.

The ear implants may each be a one piece implant that allows for a singular procedure using a singular implant, without soldering together of multiple pieces and without pore collapse resulting from melting and welding multiple ear pieces together. The ear implants may be a framework implant designed to provide reconstruction of the outer ear with natural projection, cosmesis, and improved ear symmetry. The set of ear implants may include multiple different sizes and shapes (e.g. 24 different sizes and shapes) that approximate the common range of ear shapes and sizes (with each size and shape including a left and a right configuration) providing multiple options of ear implants with different appearances to be selected from.

The ear implants may be used in total ear reconstruction of the outer ear, with the selected ear implant from the set intended to provide ear symmetry and projection to match the appearance of the contralateral ear.

In some implementations the implants may be shaped by the surgeon prior to implantation. A surgical burr or scalpel may be used to make shape adjustments to an implant. For example, a fluted burr at low RMP rinsed with sterile saline may be used to carve the implant as needed.

In one example of an operative technique, a temporoparietal fascial flap is dissected and the ear implant is positioned and oriented at the ear site symmetrically relative to the contralateral ear. The fascial flap is fixed into proper position using sutures and wrapped completely around the implant and a channel drain. Suction is applied to “shrink wrap” the flap to the implant. Sutures are used to hold the drain in place. Skin grafts are then placed over the reconstruction.

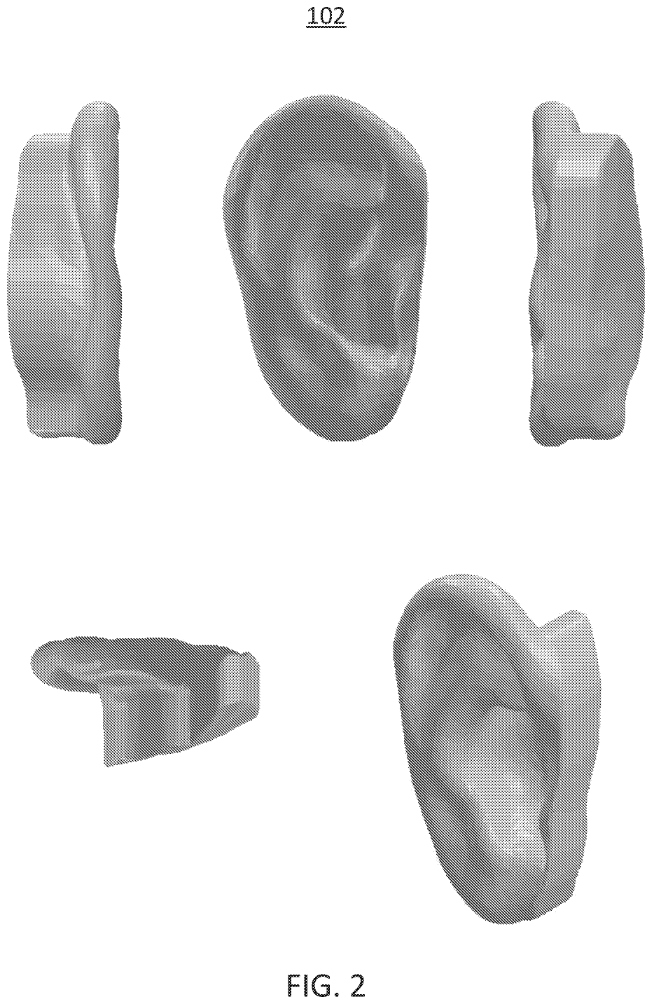

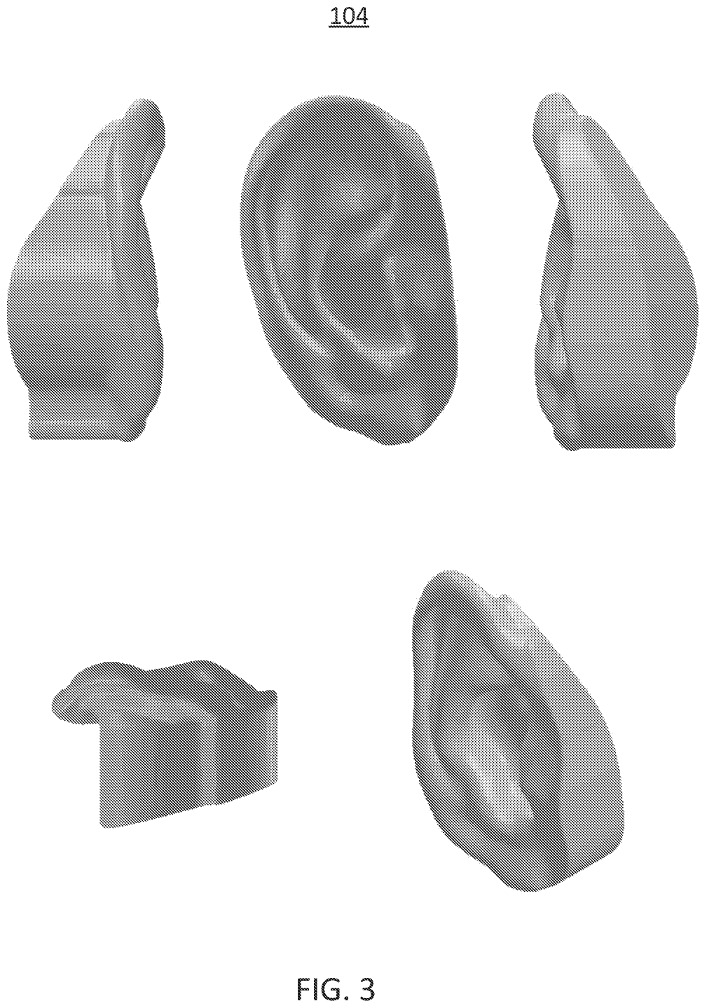

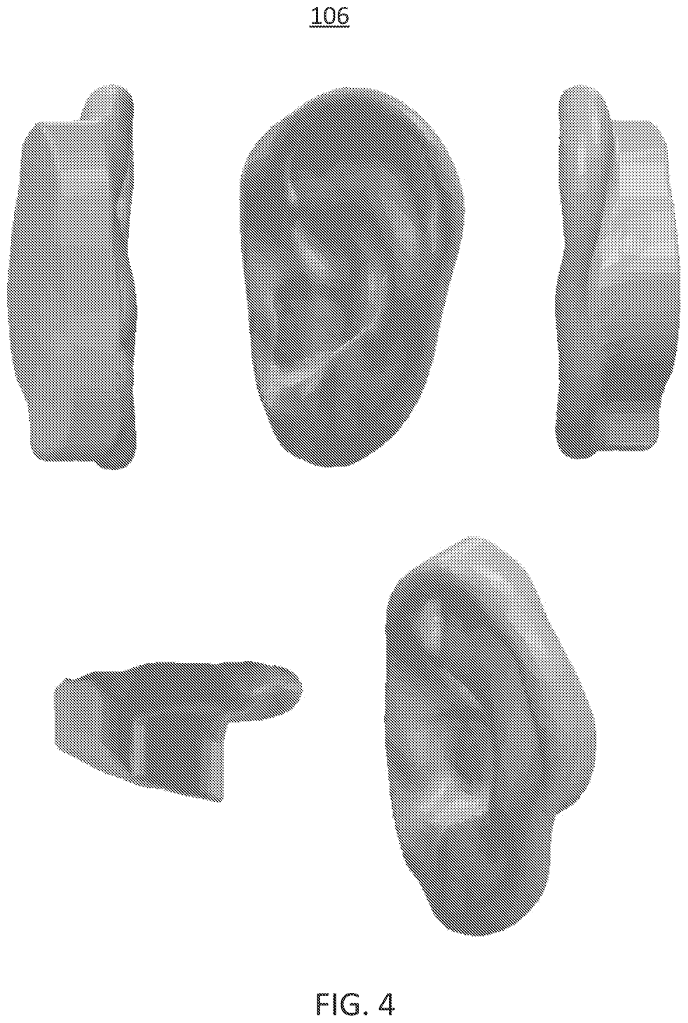

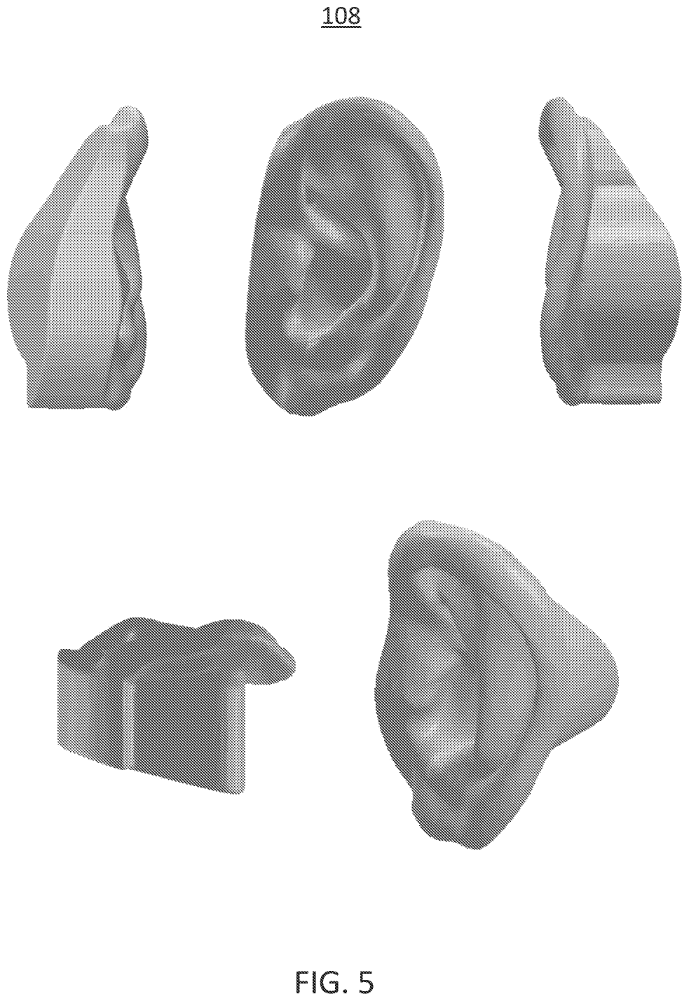

As shown in FIGS. 2-5, each of the ear implants 102, 104, 106, 108 has a particular shape, size, and appearance. The first ear implant 102 (shown in FIG. 2) has a different shape, size, and appearance from the second ear implant 104 (shown in FIG. 3). The third ear implant 106 (shown in FIG. 4) has the same shape, size, and appearance as the first ear implant 102 except that the third ear implant 106 is a contralateral version of the first ear implant 102. In this particular example, the first ear implant 102 is a right ear implant and the third ear implant 106 is a left ear implant. Similarly, the fourth ear implant 108 (shown in FIG. 5) has the same shape, size, and appearance as the second ear implant 104 (shown in FIG. 3), except that the fourth ear implant 108 is a contralateral version of the second ear implant 104. In this particular example, the second ear implant 104 is a right ear implant and the fourth ear implant 108 is a left ear implant.

The set of ear implants 100 include implants in a variety of shapes, sizes, and other aspects of appearance to allow selection of an ear implant that will closely match the shape and size (or the expected shape and size after growth) and other appearance aspects of the contralateral ear of an individual. In the example shown in FIG. 1, the set of ear implants 100 includes twenty-four ear implants, with twelve unique appearances in left and right versions. In other examples, the set of ear implants may include more or less options. In one other example, the set of ear implants may include forty-eight ear implants, with twenty-four unique appearances in left and right versions.

In the example shown, the ear implants of the set of ear implants are all one piece implants, unlike other multi-piece ear implants that are known (e.g. ear implants with separate base and helical rim components). In some implementations, the ear implants may be sintered, porous polyethylene. Exemplary polyethylenes include but are not limited to high density polyethylene or ultra high molecular weight polyethylene (UHMWPE). In other implementations, the ear implants may be hydroxyapatite (HA), expanded polytetrafluoroethylene (ePTFE), composite material such as silicone core with ePTFE outer layer, polyether ether ketone (PEEK), polyethylene terephthalate (PETE), nylon, polypropylene, or any polymer of aliphatic hydrocarbons containing one or more double bonds, or any combination thereof.

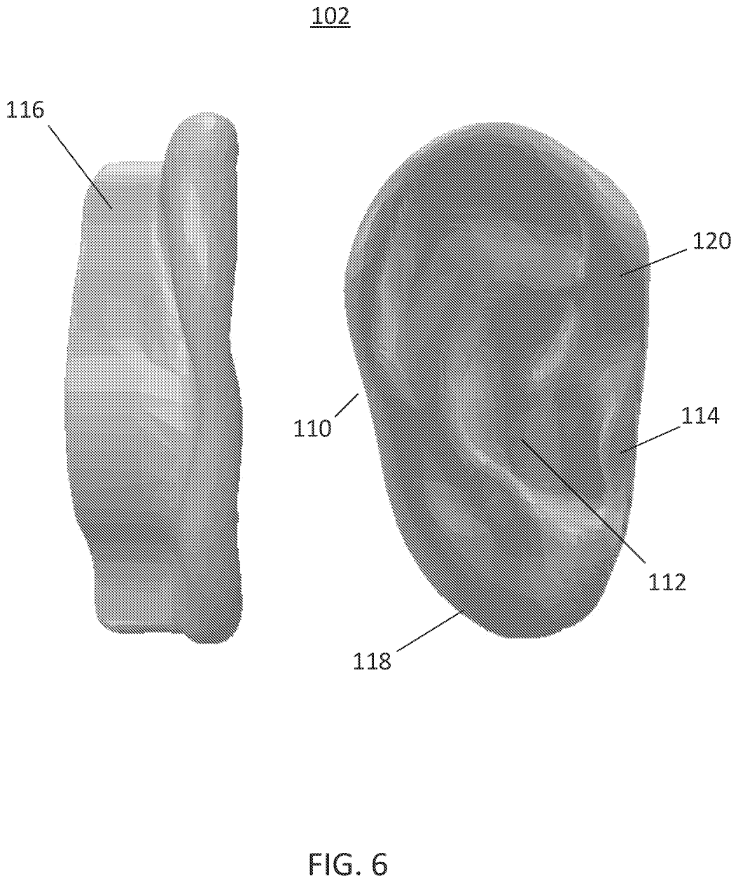

As shown in FIG. 6 the ear implants of the set of ear implants may be shaped to simulate actual outer ear anatomy. For example, the ear implant 102 in FIG. 6 include portions that simulate the helix 110, the concha 112, the tragus 114, the conchal eminence 116, the lobule 118, and the helical crus 120. The material of the ear implants (e.g. sintered polyethylene or another material) may be trimmable allowing further customization of the implant prior to implantation. For instance, in some implementation, it may be desirable to trim or otherwise reshape the conchal eminence 116 of the implant's base so that the protrusion of the implanted ear implant matches that of the contralateral ear. The set of implants provides a range that based upon a population study can be used to make nearly any desired shape of the ear needed to match the contralateral ear. While each shape may require customization by the surgeon in some implementations, it provides a selection of near net shapes that then can be modified to meet the functional and anatomical needs of the patient.

### Ear Implant Sizing Kit

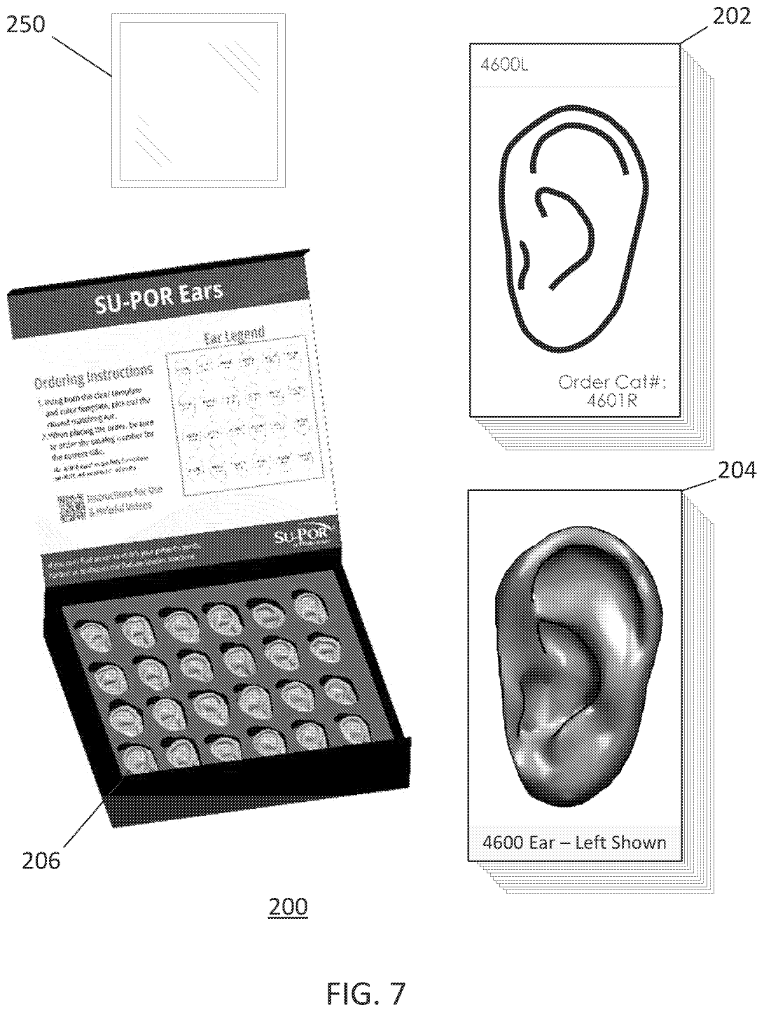

FIGS. 7-13 show an example of an ear implant sizing kit 200 that may be used to facilitate selection of an ear implant from a set of ear implants, such as the set of ear implants 100 shown in FIGS. 1-6.

In the example of FIGS. 7-13, the ear implant sizing kit 200 includes a set of semi-transparent or transparent contour templates 202, a set of gradation templates 204, a set of three-dimensional ear implant models 206, and a mirror 250 for an individual to be able to visualize what one of the three-dimensional ear implant models looks like when it is trial positioned on the individual's head.

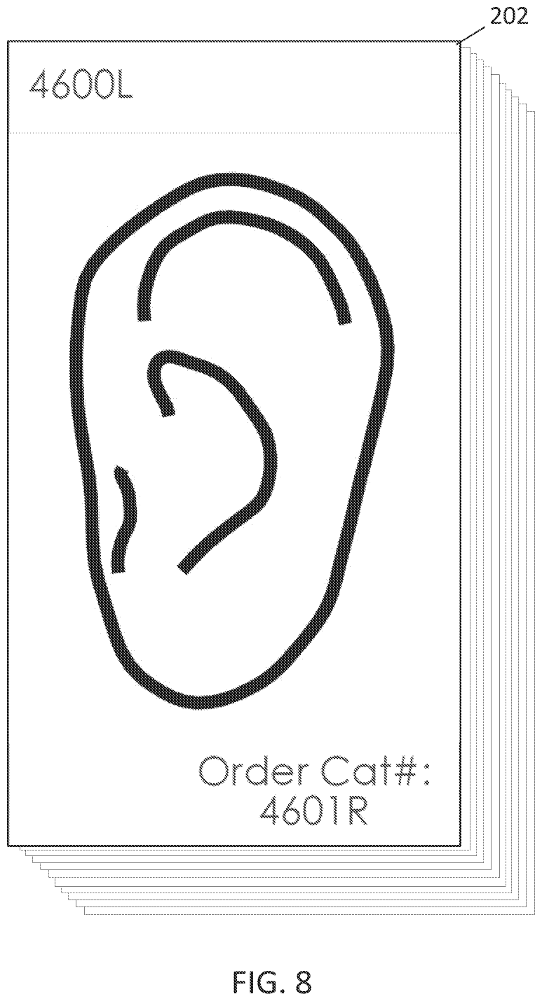

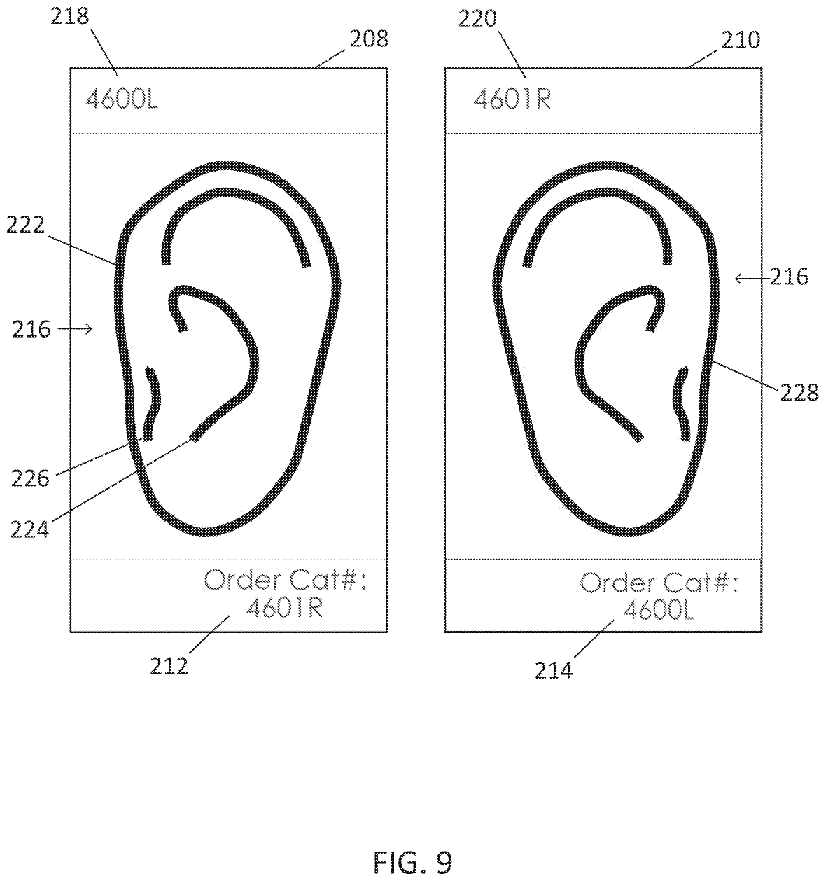

FIG. 8 shows the set of contour templates 202 and FIG. 9 shows an example of an individual contour template, with FIG. 9 including views of both a first side of the template 208 and a second side of the template 210. The contour templates 202 facilitate comparison of the contours of the various ear implants in the ear implant set 100 to the contours of an individual's contralateral ear.

The first side of the template 208 corresponds to the first ear implant 102 in the set of ear implants 100. The first side 208 includes a label 212 indicating the first side 208 is for the first ear implant 102. The second side of the template 210 corresponds to the third ear implant 106 in the set of ear implants 100 (the third ear implant 106 being the contralateral version of the first ear implant 102). The second side 210 includes a label 214 indicating the second side 210 is for the third ear implant.

The set of contour templates 202 are semi-transparent or transparent and are designed to be overlaid on the contralateral ear of an individual to assist selection of an ear implant for the individual's ipsilateral side. In this example, the first side of the template 208 shows contour markings 216 (e.g. opaque lines) that correspond in appearance to the first ear implant 102. More specifically, in this example, when viewed from the first side of the template 208, the contour markings 216 are mirror images of corresponding contours of the first ear implant 102, facilitating comparison with the contralateral ear to the side of the patient's head where the implant will be implanted. In other words, the first side of the template 208 is for selecting the first ear implant 102 but shows contours corresponding in appearance to a contralateral version of the first ear implant 102. The additional label 218 on the first side 208 indicates the contralateral version of the first ear implant 102 (e.g. the third ear implant 106).

In this example, when the template is viewed from the second side 210, the contour markings 216 correspond in appearance to the third ear implant 106. When viewed from the second side 210, the contour markings 216 are mirror images of corresponding contours of the third ear implant 102. In other words, the second side of the template 210 is for selecting the third ear implant 106 but shows contours corresponding in appearance to a contralateral version of the third ear implant 106. The second side 210 of the template includes an additional label 220 indicating the contralateral version of the third ear implant 106 (e.g. the first ear implant 102).

When viewed from the first side of the contour template 208, the contour markings 216 correspond in size, shape, and appearance to an outer perimeter of the ear implant shown in FIG. 6. The contour markings 216 also include a contour marking portion 222 corresponding to a size, shape, and appearance of a helix 110 of the ear implant 102 shown in FIG. 6 and also include contour marking portions 224, 226 corresponding to sizes, shapes, and appearances of a concha 112 and a tragus 114 of the FIG. 6 ear implant 102 respectively. Again, although the marking portions 222, 224, and 226 correspond in size, shape, and appearance to the portions 110, 112, and 114 of ear implant 102, those marking portions, when viewed from the first side 208, are mirror images of the implant portions they correspond to. Additionally, as can be seen by comparing FIG. 9 to FIG. 6, although the marking portions 222, 224, and 226 correspond in size, shape, and appearances to the portions 110, 112, and 114 of ear implant 102, they do not necessarily completely replicate those portions of the ear implant 102 in their entireties. For example, the helix contour marking portion 222 does not extend the entire length of the helix 110 of the ear implant 102.

When viewed from the second side of the contour template 210, the contour markings 216 correspond in size, shape, and appearance to an outer perimeter of the ear implant 106 (the contralateral version of ear implant 102). The contour markings 216 also include a contour marking portion 228 corresponding to a size, shape, and appearance of a helix of the ear implant 106 and also include contour marking portions corresponding to sizes, shapes, and appearances of a concha and tragus of the ear implant 106 respectively.

While in this example the contour templates use contour markings corresponding to the helix, concha, and tragus, in other implementations, other ear anatomy may be used additionally or instead of the helix, concha, and/or tragus.

In the example shown, the templates 202 are referred to as semi-transparent or transparent because the portions of the templates 202 including contour markings 216 are semi-transparent or transparent, but other portions of the templates 202 (e.g. the portions with labels 212, 214, 218, 220 may be partially or completely opaque).

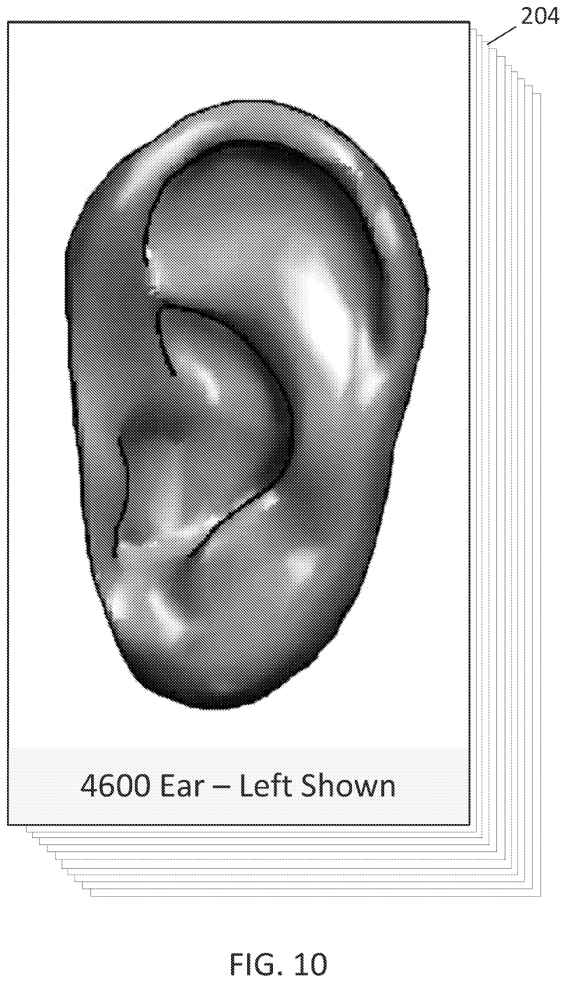

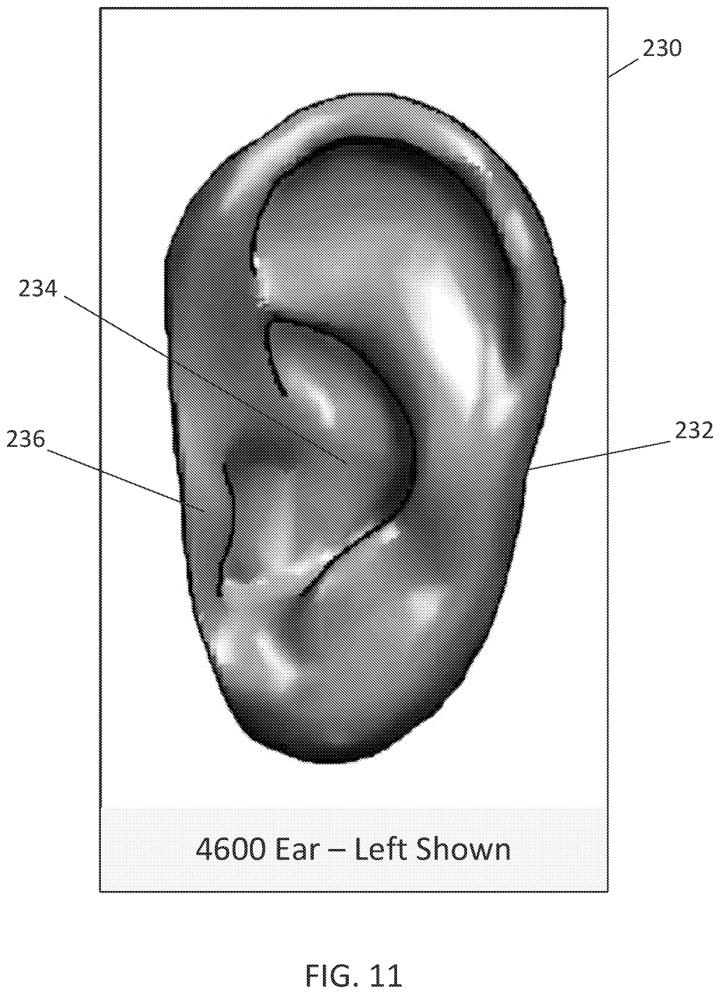

FIG. 10 shows the set of gradation templates 204 and FIG. 11 shows an example of an individual gradation template 230, which in this example corresponds in appearance to the ear implant 106 shown in FIG. 1. Each of the gradation templates in the set 204 includes depth shading that corresponds in appearance to feature depth of its corresponding implant. For instances, in the example shown in FIG. 11, helix 232, concha 234, and tragus 236 portions on the template 230 are all shaded to help visualize the depth of the features for the corresponding implant. In this example the darkness or lightness of the shading indicates the relative depth of a feature or a portion of a feature relative to other features or portions of features. In other implementations, other shading techniques may be utilized to indicate the relative depth of features and portions of features.

In this particular example, the set of gradation templates 204 does not include both left and right versions corresponding to the left and right versions of the implants. For instance, the set of gradation templates 204 may only show left versions of the ear implants, and not include templates for the right versions of the ear implants.

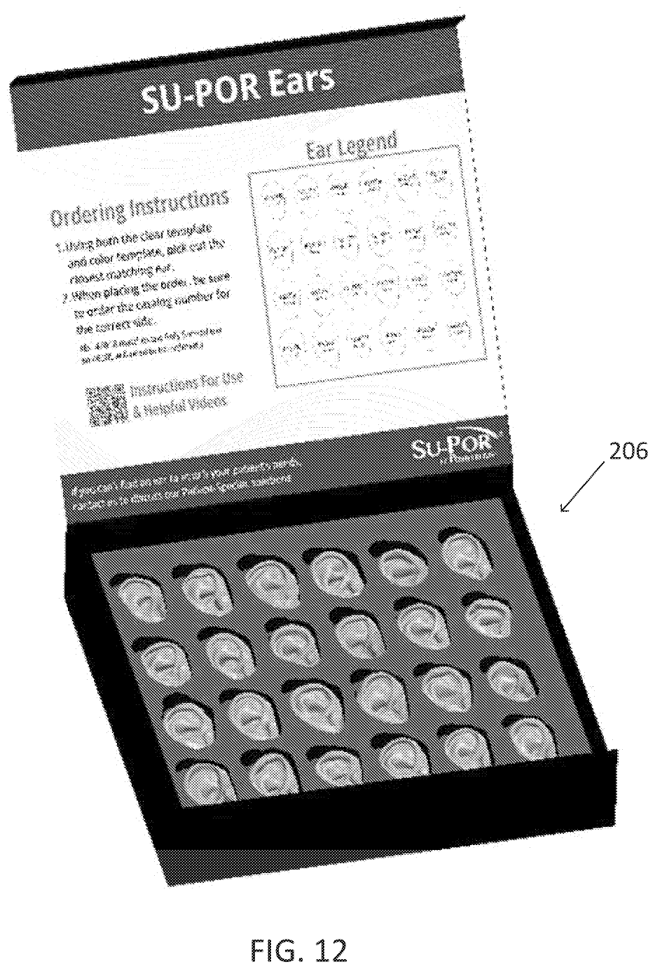

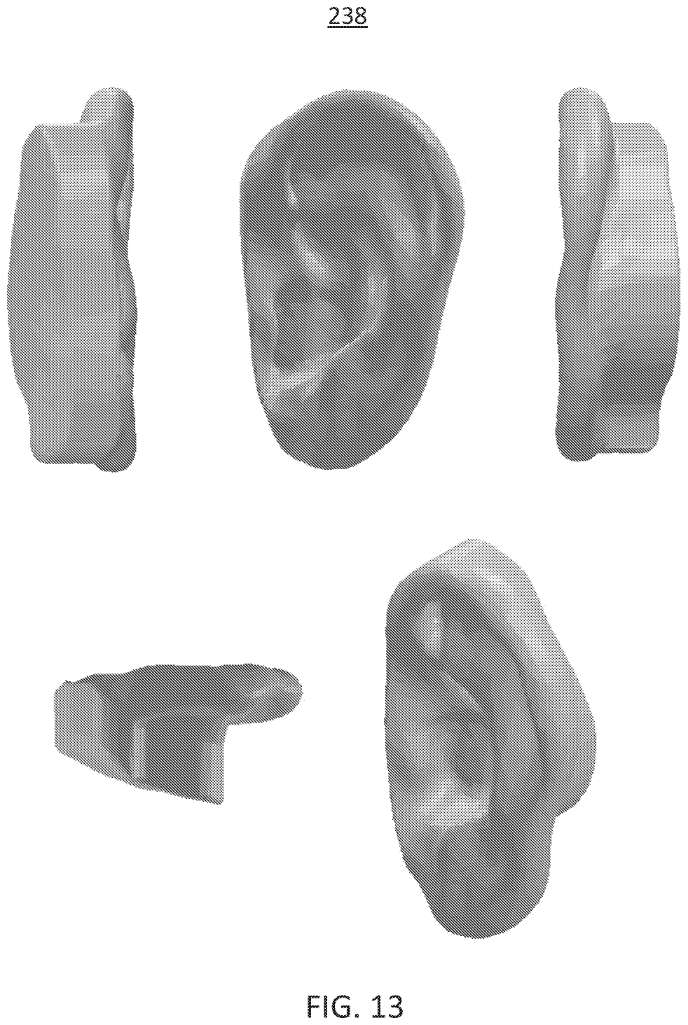

FIG. 12 shows the set of three-dimensional ear implant models 206 and FIG. 13 shows an example on individual three-dimensional ear implant model 238 from multiple views. In this example, the three-dimensional ear implant model 238 corresponds in appearance to the ear implant 106 shown in FIG. 1. In this example, the three-dimensional ear implant model 238 has the same appearance and the same three-dimensional size and three-dimensional shape as the ear implant 106 shown in FIG. 1. In this particular example, as with the set of gradation templates 204, the set of three-dimensional ear implant models 206 does not include both left and right versions corresponding to the left and right versions of the implants (e.g. the set may include only the left versions and not the right versions).

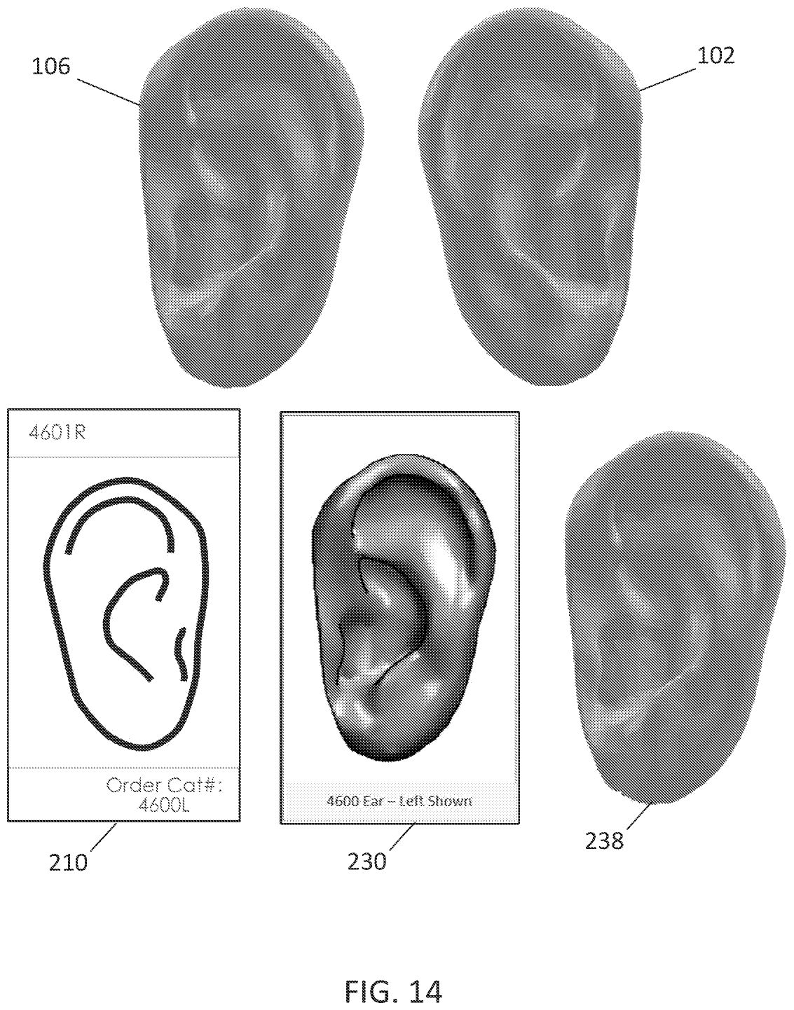

FIG. 14 shows the components of an ear implant set 100 and ear implant selection kit 200 corresponding in appearance to the ear implant set 100. In this example, that includes: (1) the first ear implant 102 (e.g. a right ear implant) and the third ear implant 106 (e.g. a left ear implant that is a contralateral version of the first ear implant); (2) the contour template corresponding in appearance to the ear implant set that includes a second side 210 corresponding to the third implant 106 and a first side (not shown) corresponding to the first implant 102; (3) the gradation template 230 corresponding in appearance to the ear implant set (with the left ear implant being shown); and (4) the ear implant model 238 corresponding in appearance to the ear implant set (and being a replica of the left ear implant). As noted earlier, and as illustrated by FIG. 14, the gradation templates and the three dimensional models will not necessarily be in the kit 200 in both left and right versions.

### Method of Selecting an Ear Implant

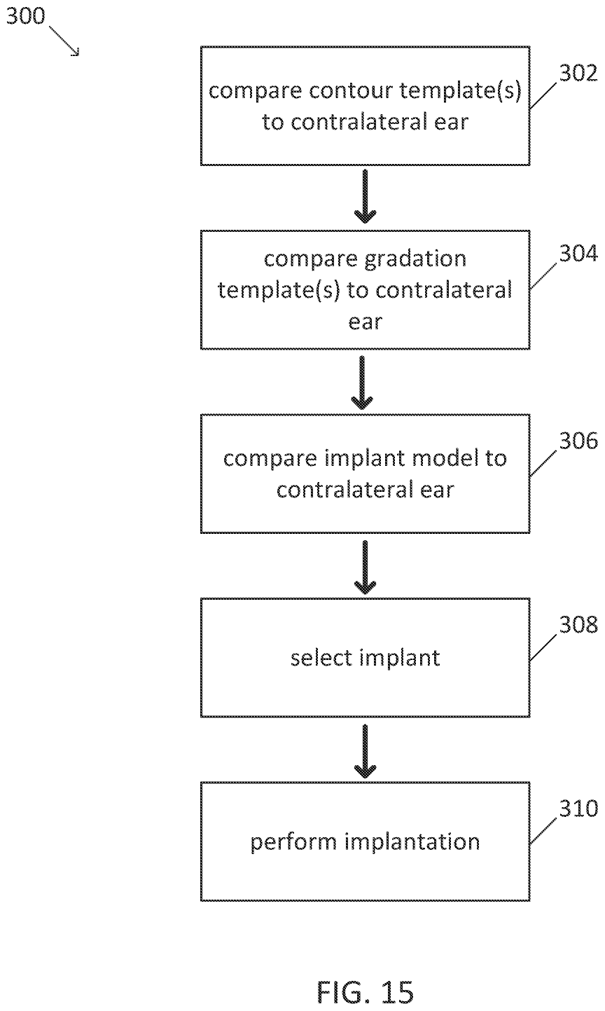

FIG. 15 summarizes an example of a method 300 of selecting an ear implant from an ear implant set. The method of FIG. 15 may use the ear implant set 100 and ear implant selection kit 200 described above or may use other implant sets and sizing kits.

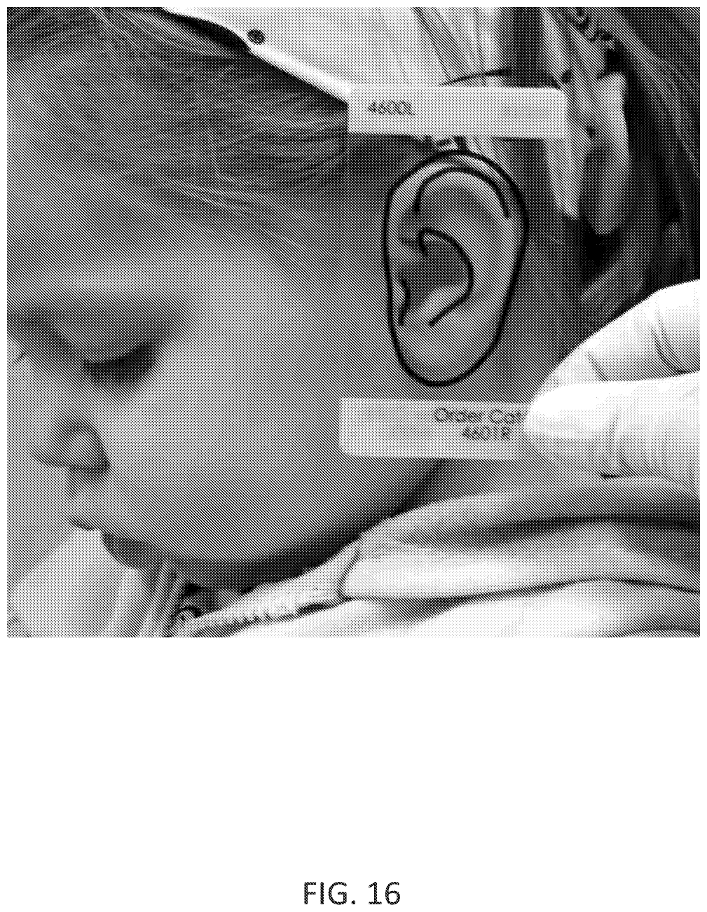

At step 302, using an ear implant selection kit, at least one template of a set of semi-transparent or transparent contour templates is compared to a contralateral ear of an individual. FIG. 16 shows an example of comparing a semi-transparent or transparent contour template to a contralateral ear of an individual. The contour templates each correspond in appearance to a particular appearance of one of the ear implants in the set of ear implants.

At step 304, using the ear implant selection kit, at least one gradation template of a set of gradation templates is compared to the contralateral ear of the individual. The gradation templates each correspond in appearance to a particular appearance of one of the ear implants in the set of ear implants.

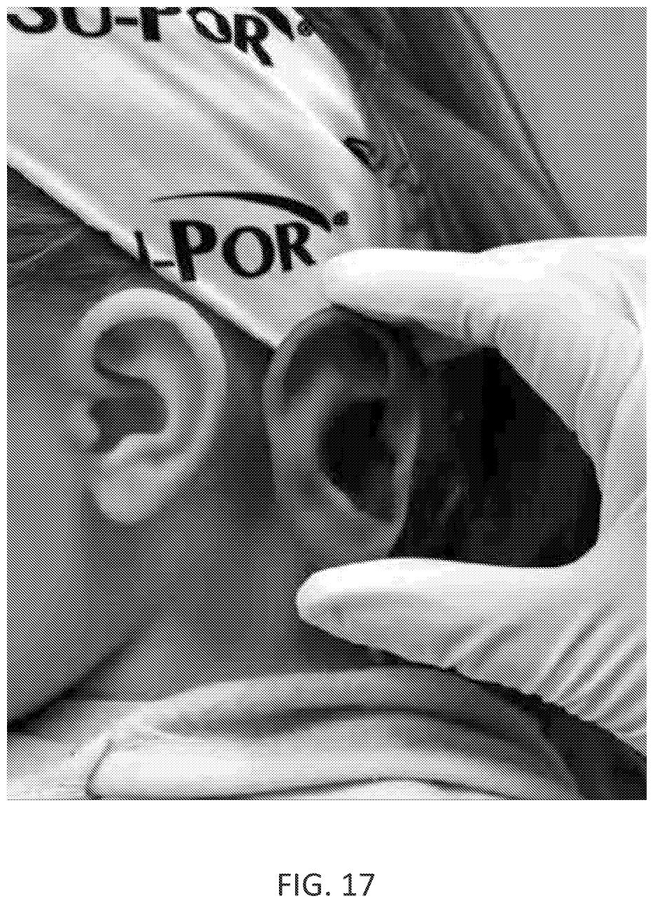

At step 306, using the ear implant selection kit, at least one three-dimensional implant model of a set of three-dimensional implant models is compared to the contralateral ear of the individual. The three-dimensional implant models each correspond in appearance to a particular appearance of one of the ear implants in the set of ear implants. FIG. 17 shows an example comparing a three-dimensional implant model to the contralateral ear of the individual.

At step 308, based on these comparisons, a particular ear implant is selected out of a set of ear implants. In some implementations, labels on or otherwise associated with the contour templates, gradation templates, and/or three-dimensional implant models may be used to identify the particular ear implant for ordering the implant.

At step 310, the selected ear implant may be implanted as described earlier.

Additions, deletions, substitutions, and other alterations or modifications may be made to the systems, kits, and methods described above without departing from the scope or the spirit of the presently claimed inventions, which are set out in the following claims.

1. An outer ear implant selection kit for selecting an outer ear implant from a set of outer ear implants, the set comprising at least a first outer ear implant comprising a first appearance and a second outer ear implant comprising a second appearance, wherein the first appearance is different from the second appearance, the outer ear implant selection kit comprising:
(a) a set of templates corresponding to the set of outer ear implants, the set of templates comprising at least a first template and a second template;
(b) wherein the first template corresponds in appearance to the first outer ear implant; and
(c) wherein the second template corresponds in appearance to the second outer ear implant.

2. The outer ear selection kit of claim 1 further comprising a set of three-dimensional ear implant models comprising a first three-dimensional outer ear implant model that corresponds in appearance to the first outer ear implant and a second three-dimensional outer ear implant model that corresponds in appearance to the second outer ear implant.

3. The outer ear selection kit of claim 2 wherein the set of templates comprises a set of semi-transparent or transparent contour templates, wherein the first template comprises a first contour template comprising a first side corresponding in appearance to the first outer ear implant, wherein the second template comprises a second contour template comprising a first side corresponding in appearance to the second outer ear implant.

4. The outer ear selection kit of claim 3 wherein the first contour template includes a label for the first outer ear implant and the second contour template includes a label for the second outer ear implant.

5. The outer ear selection kit of claim 3 wherein the first contour template comprises first contour markings that, from the first side of the first contour template, correspond in appearance to contours of the first outer ear implant; and the second contour template comprises second contour markings that are different from the first contour markings, the second contour markings, from the first side of the second contour template, correspond in appearance to contours of the second outer ear implant.

6. The outer ear selection kit of claim 5 wherein the contour markings of the first and second contour templates are opaque or semi-opaque contour lines.

7. The outer ear selection kit of claim 3, wherein the set of outer ear implants further comprises a third outer ear implant that is a contralateral version of the first outer ear implant and a fourth outer ear implant that is a contralateral version of the second outer ear implant, wherein the ear selection kit further comprises:
(a) a second side of the first contour template, wherein the contour markings of the first contour template, from the second side of the first contour template, correspond in appearance to the third outer ear implant; and
(b) a second side of the second contour template, wherein the contour markings of the second contour template, from the second side of the second contour template, correspond in appearance to the fourth outer ear implant.

8. The outer ear selection kit of claim 7 wherein:
(a) the first side of the first contour template is labeled for the first outer ear implant and the second side of the first contour template is labeled for the third outer ear implant; and
(b) the first side of the second contour template is labeled for the second outer ear implant and the second side of the second contour template is labeled for the fourth outer ear implant.

9. The outer ear selection kit of claim 3 wherein:
(a) the contour markings of the first contour template comprise an outer perimeter shape corresponding in appearance to an outer perimeter shape of the first outer ear implant; and
(b) the contour markings of the second contour template comprise an outer perimeter shape corresponding in appearance to an outer perimeter shape of the second outer ear implant.

10. The outer ear selection kit of claim 9 wherein:
(a) the contour markings of the first contour template further comprise at least one of: (i) a first helix contour marking corresponding in appearance to the first outer ear implant; (ii) a first concha contour marking corresponding in appearance to the first outer ear implant;
and (iii) a first tragus contour marking corresponding in appearance to the first outer ear implant; and
(b) the contour markings of the second contour template further comprise at least one of (i) a second helix contour marking corresponding in appearance to the second outer ear implant; (ii) a second concha contour marking corresponding in appearance to the second outer ear implant; and (iii) a second tragus contour marking corresponding in appearance to the second outer ear implant.

11. The outer ear selection kit of claim 3 further comprising a set of gradation templates comprising: a first gradation template corresponding in appearance to the first outer ear implant, the first gradation template being labeled for the first outer ear implant; and a second gradation template corresponding in appearance to the second outer ear implant, the second gradation template being labeled for the second ear implant.

12. The outer ear selection kit of claim 11 wherein:
(a) the first gradation template comprises first depth shading that corresponds in appearance to the first outer ear implant; and
(b) the second gradation template comprises second depth shading that is different from the first depth shading and that corresponds in appearance to the second outer ear implant.

13. The outer ear selection kit of claim 12 wherein:
(a) the first depth shading of the first gradation template comprises at least one of: (i) a first helix depth shading corresponding in appearance to a helix of the first outer ear implant;
(ii) a first concha depth shading corresponding in appearance to a concha of the first outer ear implant; and (iii) a first tragus depth shading corresponding in appearance to a tragus of the first outer ear implant; and
(b) the second depth shading of the second gradation template comprises at least one of: (i) a second helix depth shading corresponding in appearance to a helix of the second outer ear implant; (ii) a second concha depth shading corresponding in appearance to a concha of the second outer ear implant; and (iii) a second tragus depth shading corresponding in appearance to a tragus of the second outer ear implant.

14. The outer ear selection kit of claim 1 wherein:
(a) the first outer ear implant comprises a one piece implant comprising a tragus, a concha, a helix, and a conchal eminence;
(b) the second outer ear implant comprises a one piece implant comprising a tragus, a concha, a helix, and a conchal eminence; and
(c) wherein an appearance of at least one of the tragus, concha, and helix of the first outer ear implant is different from an appearance of the tragus, concha, and helix of the second outer ear implant.

15. The outer ear selection kit of claim 14 wherein the set of outer ear implants is a set of outer ear framework implants.

16. A method of selecting an outer ear implant for implantation on a person from a set of outer ear implants, the set of outer ear implants comprising a plurality of outer ear implants having different aesthetic appearances, the method of selecting from the set of outer ear implants comprising:
(a) using a set of templates comprising a plurality of templates each corresponding in appearance to one of the plurality of outer ear implants, comparing at least one of the templates from the set of templates to a contralateral ear of the person; and
(b) based on the comparison of the at least one template, selecting one of the outer ear implants from the set of outer ear implants for ipsilateral implantation on the person.

17. The method of selecting an outer ear implant of claim 16 further comprising:
(a) using a set of three-dimensional ear implant models each corresponding in appearance to one of the plurality of outer ear implants, trialing at least one of the three-dimensional ear implant models with the person; and
(b) based on the comparison of the at least one template and based on the trialing of the at least one three-dimensional ear implant model, selecting one of the outer ear implants from the set of outer ear implants for ipsilateral implantation on the person.

18. The method of selecting an outer ear implant of claim 17 wherein the set of templates comprises a set of semi-transparent or transparent contour templates, and wherein comparing the at least one template to the contralateral ear of the person comprises positioning the at least one contour template over the contralateral ear of the person.

19. The method of selecting an outer ear implant of claim 18 wherein the at least one contour template positioned over the contralateral ear of the person comprises contour markings indicating an outer perimeter shape corresponding in appearance to an outer perimeter shape of a first outer ear implants of the set of outer ear implants.

20. The method of selecting an outer ear implant of claim 19 wherein the at least one contour template positioned over the contralateral ear of the person further comprises additional contour markings comprising at least one of: (i) a helix contour marking corresponding in appearance to the first outer ear implant; (ii) a first concha contour marking corresponding in appearance to the first outer ear implant; and (iii) a first tragus contour marking corresponding in appearance to the first outer ear implant.

21. The method of selecting an outer ear implant of claim 18 further comprising:
(a) using a set of gradation templates comprising a plurality of gradation templates each corresponding in appearance to one of the plurality of outer ear implants, comparing at least one of the gradation templates to the contralateral ear of the person; and
(b) based on the comparison of the at least one contour template and based on the trialing of the at least one three-dimensional ear implant model and based on the comparison of the at least one gradation template, selecting one of the outer ear implants from the set of outer ear implants for ipsilateral implantation on the person.

22. The method of selecting an outer ear implant of claim 16, wherein the outer ear implants of the set of outer ear implants each comprises a one piece implant comprising a tragus, a concha, a helix, and a conchal eminence.

23. The method of selecting an outer ear implant of claim 22 wherein the outer ear implants of the set of outer ear are each an outer ear framework implant.
